(19) United States
(12) Patent Application Publication
(10) Pub. No.: US 2026/0245736 A1
(43) Pub. Date: Aug. 20, 2026

(54) METHOD AND SYSTEM FOR PREDICTING LIVER ASSOCIATED DISEASE

(71) Applicants: Yeda Research and Development Co. Ltd., Rehovot (IL); Mor Research Applications Ltd., Ramat Gan (IL)

(72) Inventors: Eran SEGAL, Ramat-HaSharon (IL); Adina WEINBERGER, Rehovot (IL); Iris KALKA, Rehovot (IL); Ziv NEEMAN, Ramat HaSharon (IL); Rawi HAZZAN, Nof HaGalil (IL)

(73) Assignees: Yeda Research and Development Co. Ltd., Rehovot (IL); Mor Research Applications Ltd., Ramat Gan (IL)

(21) Appl. No.: 19/472,754

(22) PCT Filed: Apr. 4, 2024
(86) PCT No.: PCT/IL2024/050348

§ 371 Date: Oct. 6, 2025

(30) Foreign Application Priority Data
Apr. 4, 2023 (IL) 301945

(51) Int. Cl. G16H 50/30

(52) U.S. Cl. CPC G16H 50/30

(57) ABSTRACT

A system for predicting probability for developing a liver associated disease, comprises a data processor which obtains parameters extracted from a body liquid test applied to a healthy subject, and feeds a machine learning procedure with the parameters. The machine learning procedure is trained for predicting probabilities for liver associated disease, and the processor receives from procedure an output indicative of a probability that the subject is expected to develop a liver associated disease.

### RELATED APPLICATION

This application claims the benefit of priority of Israeli Patent Application No. 301945 filed on Apr. 4, 2023, the contents of which are incorporated herein by reference in their entirety.

### FIELD AND BACKGROUND OF THE INVENTION

The present invention, in some embodiments thereof, relates to medicine and, more particularly, but not exclusively, to a method and system for predicting liver associated disease.

Liver diseases account for approximately two-million annual deaths worldwide, half due to liver cirrhosis complications. Diagnostic tests include biopsy and are performed mainly in high-risk populations. Also known are non-invasive tests such as MRI elastography, transient elastography, and ultrasound elastography, directed to provide tissue characteristic information, such as elastic modulus, viscosity or stiffness. Further known are specific blood tests such as the Fibrosis-4 test (FIB-4) and the non-alcoholic fatty liver disease fibrosis score [Angulo et al., Hepatology 2007; 45 (4): 846-54, Sterling et al., Hepatology 2006; 43 (6): 1317-25].

Hanif et al., 2022 IEEE 13th Annual Ubiquitous Computing, Electronics & Mobile Communication Conference (UEMCON), 2022, pp. 0028-0034, doi: 10.1109/UEMCON54665.2022.9965718, discloses application of an artificial intelligence system to a previously prepared digital dataset for the prediction of liver cirrhosis.

International patent application No. WO2021/091288, discloses a machine learning model for predicting hepatocellular carcinoma in subjects diagnosed with hepatitis B.

Chinese patent application No. CN112669960A discloses applying machine learning to biopsies for the prediction of liver fibrosis.

Korean Patent No. KR100999720B1 discloses an analytical method for diagnosing liver cirrhosis, by performing blood tests employing serological markers including serum 2-macroglobulin, serum apolipoprotein A1, and serum hyaluronic acid (HA).

### SUMMARY OF THE INVENTION

According to an aspect of some embodiments of the present invention there is provided a system for predicting probability for developing a liver associated disease, the system comprising a data processor having a circuit configured to obtain a plurality of parameters extracted from a body liquid test applied to a healthy subject, to access a computer readable medium storing a machine learning procedure trained for predicting probabilities for liver associated disease, to feed the procedure with the plurality of parameters, and to receive from the procedure an output indicative of a probability that the subject is expected to develop a liver associated disease.

According to some embodiments of the invention the circuit is configured to receive from the procedure an output indicative of an expected onset time of the liver associated disease in the subject.

According to some embodiments of the invention the plurality of parameters comprises a blood level of aspartate aminotransferase and a platelet score, and is devoid of blood hyaluronic acid concentration, and blood procollagen III-NP concentration.

According to an aspect of some embodiments of the present invention there is provided a system for predicting a response to a pharmacological agent for treating a liver associated disease, the system comprising data processor having a circuit configured to obtain a plurality of parameters extracted from a body liquid test applied to a subject administered the pharmacological agent, to access a computer readable medium storing a machine learning procedure trained for predicting probabilities for liver associated disease, to feeding the procedure with the plurality of parameters, to receive from the procedure an output indicative of a probability that the subject is expected to develop a liver associated disease, and to generate an output indicative of the response to the pharmacological agent based on the probability, wherein the plurality of parameters comprises a blood level of aspartate aminotransferase and a platelet score, and is devoid of blood hyaluronic acid concentration, and blood procollagen III-NP concentration.

According to an aspect of some embodiments of the present invention there is provided a method of predicting probability for developing a liver associated disease, comprising: obtaining a plurality of parameters extracted from a body liquid test applied to a healthy subject; accessing a computer readable medium storing a machine learning procedure trained for predicting probabilities for liver associated disease; feeding the procedure with the plurality of parameters; and receiving from the procedure an output indicative of a probability that the subject is expected to develop a liver associated disease.

According to some embodiments of the invention the method comprises when the probability is above a predetermined threshold, performing at least one imaging test directed to identify the liver associated disease.

According to some embodiments of the invention the imaging test comprises elastography.

According to some embodiments of the invention the method comprises when the probability is above a predetermined threshold, periodically monitoring the subject for onset of the liver associated disease, by at least one technique selected from the group consisting of biopsy, MRI elastography, transient elastography, ultrasound elastography, and a fibrosis test

According to some embodiments of the invention the fibrosis test is more preferably fibrosis-4 (FIB-4), aspartate aminotransferase to platelet ratio index (APRI), and aspartate aminotransferase to alanine aminotransferase ratio.

According to some embodiments of the invention the fibrosis test comprises fibrosis-4 (FIB-4), aspartate aminotransferase to platelet ratio index (APRI), and aspartate aminotransferase to alanine aminotransferase ratio.

According to some embodiments of the invention the fibrosis test is consisting of fibrosis-4 (FIB-4), aspartate aminotransferase to platelet ratio index (APRI), and aspartate aminotransferase to alanine aminotransferase ratio.

According to some embodiments of the invention the method comprises receiving from the procedure an output indicative of an expected onset time of the liver associated disease in the subject.

According to some embodiments of the invention the method comprises, a predetermined time ahead of the expected onset time, periodically monitoring the subject for onset of the liver associated disease, by at least one technique selected from the group consisting of biopsy, MRI elastography, transient elastography, ultrasound elastography, and a fibrosis test.

According to some embodiments of the invention the plurality of parameters comprises a blood level of aspartate aminotransferase and a platelet score, and is devoid of blood hyaluronic acid concentration, and blood procollagen III-NP concentration.

According to some embodiments of the invention the method comprises applying measures to prevent a development of the liver associated disease or the reduce the probability.

According to some embodiments of the invention the measures comprise administering a pharmaceutical agent. According to some embodiments of the invention the measures comprise administering nutritional supplements or a dietary formulation. According to some embodiments of the invention the measures comprise providing recommendations for a change of life style and/or dietary habits, including, but not limited to, liver cirrhosis diet. The dietary habits may include maintaining lean body mass, prioritizing plant-base and animal-based protein sources, avoiding or limiting alcohol consumption and staying hydrated.

According to an aspect of some embodiments of the present invention there is provided a computer software product, comprises a computer-readable medium in which program instructions are stored, which instructions, when read by a data processor, cause the data processor to receive a food to which a response of a subject is unknown, and to execute the method as delineated above and optionally and preferably as further detailed below.

According to an aspect of some embodiments of the present invention there is provided a method of evaluating efficacy of a treatment for a liver associated disease, comprising: obtaining a plurality of parameters extracted from a body liquid test applied to a subject undiagnosed with any liver associated condition; accessing a computer readable medium storing a machine learning procedure trained for predicting probabilities for liver associated disease; feeding the procedure with the plurality of parameters; receiving from the procedure an output indicative of a probability that the subject is expected to develop a liver associated disease; treating the subject by the treatment to evaluate the efficacy thereof only if the probability is above a predetermined threshold.

According to an aspect of some embodiments of the present invention there is provided a method of predicting a response to a pharmacological agent for treating a liver associated disease, comprising: obtaining a plurality of parameters extracted from a body liquid test applied to a subject administered the pharmacological agent, wherein the plurality of parameters comprises a blood level of aspartate aminotransferase and a platelet score, and is devoid of blood hyaluronic acid concentration, and blood procollagen III-NP concentration; accessing a computer readable medium storing a machine learning procedure trained for predicting probabilities for liver associated disease; feeding the procedure with the plurality of parameters; and receiving from the procedure an output indicative of a probability that the subject is expected to develop a liver associated disease, and generating an output indicative of the response to the pharmacological agent based on the probability.

According to some embodiments of the invention the plurality of parameters comprises at least one parameter extracted from an electronic health record associated with the subject.

According to some embodiments of the invention the plurality of parameters comprises at least one parameter extracted from a body liquid test applied to the subject.

According to some embodiments of the invention the plurality of parameters comprises at least five blood levels or counts selected from the group consisting of: hemoglobin, platelet, white blood count, AST (GOT), ALT (GPT), albumin, bilirubin total, PT-INR, vitamin B12, glucose, hemoglobin A1c, cholesterol, cholesterol—HDL, cholesterol—LDL calc, triglycerides, and protein—total.

According to some embodiments of the invention the plurality of parameters comprises blood level of aspartate aminotransferase, platelet score, an age of the subject, blood level of albumin, and blood level of glucose.

According to some embodiments of the machine learning procedure comprises at least one procedure selected from the group consisting of clustering, support vector machine, linear modeling, k-nearest neighbors analysis, a set of decision trees, ensemble learning procedure, neural networks, probabilistic model, graphical model, Bayesian network, and association rule learning.

According to some embodiments of the machine learning procedure comprises a set of decision trees.

According to some embodiments of the invention the set of decision trees is trained by gradient boosting.

According to some embodiments of the liver associated disease comprises liver cirrhosis.

According to some embodiments of the liver associated disease comprises at least one of: liver fibrosis, nonalcoholic fatty liver disease, non-alcoholic steatohepatitis, alcoholic fatty liver disease, alcoholic steatohepatitis, autoimmune hepatitis, hepatocarcinoma.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration showing an overview of a retrospective cohort used in experiments performed according to some embodiments of the present invention;

FIG. 2 shows true positive rate as a function of false positive rate, as obtained for a retrospective cohort in experiments performed according to some embodiments of the present invention;

FIG. 3 is an illustration showing an overview of a prospective cohort used in experiments performed according to some embodiments of the present invention;

FIG. 4 shows liver stiffness in a clinical cohort, as obtained in experiments performed according to some embodiments of the present invention;

FIGS. 5A and 5B show percentage of individuals who halted follow-up per year post index-date, as obtained in experiments performed according to some embodiments of the present invention;

FIGS. 6A and 6B show counts (FIG. 6A) and percentage (FIG. 6B) of liver stiffness in the prospective cohort, as obtained in experiments performed according to some embodiments of the present invention;

FIG. 7 shows AUDIT scores for individuals separated by groups, as obtained in experiments performed according to some embodiments of the present invention;

FIG. 8 is a flowchart diagram of a method suitable for predicting probability for developing a liver associated disease, according to various exemplary embodiments of the present invention;

FIG. 9 is a flowchart diagram of a method suitable for evaluating efficacy of a treatment for a liver associated disease, according to some embodiments of the present invention;

FIG. 10 is a flowchart diagram of a method suitable for predicting a response to a treatment, according to some embodiments of the present invention; and

FIG. 11 is a schematic illustration of a computing platform that can be used for executing one or more of the methods described herein.

### DESCRIPTION OF SPECIFIC EMBODIMENTS OF THE INVENTION

The present invention, in some embodiments thereof, relates to medicine and, more particularly, but not exclusively, to a method and system for predicting liver associated disease.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

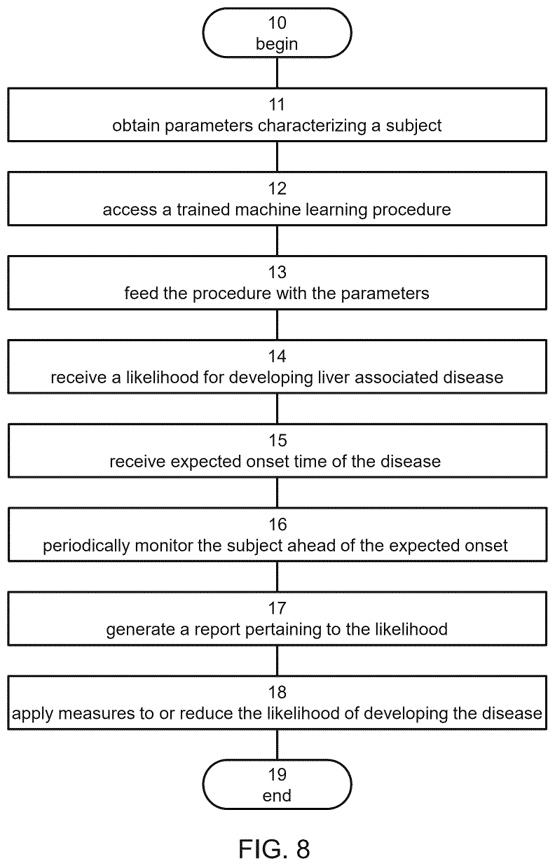

FIG. 8 is a flowchart diagram of a method suitable for predicting probability for developing a liver associated disease, according to various exemplary embodiments of the present invention.

It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The processing operations of the present embodiments can be embodied in many forms. For example, they can be embodied in on a tangible medium such as a computer for performing the operations. They can be embodied on a computer readable medium, comprising computer readable instructions for carrying out the method operations. They can also be embodied in electronic device having digital computer capabilities arranged to run the computer program on the tangible medium or execute the instruction on a computer readable medium.

Computer programs implementing the method according to some embodiments of this invention can commonly be distributed to users on a distribution medium such as, but not limited to, CD-ROM, flash memory devices, flash drives, or, in some embodiments, drives accessible by means of network communication, over the internet (e.g., within a cloud environment), or over a cellular network. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. Computer programs implementing the method according to some embodiments of this invention can also be executed by one or more data processors that belong to a cloud computing environment. All these operations are well-known to those skilled in the art of computer systems. Data used and/or provided by the method of the present embodiments can be transmitted by means of network communication, over the internet, over a cellular network or over any type of network, suitable for data transmission.

The method can be used for predicting any liver associated disease, including, without limitation, liver cirrhosis, liver fibrosis, non-alcoholic fatty liver disease (NAFLD), non-alcoholic steatohepatitis (NASH), alcoholic steatohepatitis (ASH), hepatic ischemia reperfusion injury, primary biliary cirrhosis (PBC), primary sclerosing cholangitis (PSC), and hepatitis, including both viral and alcoholic hepatitis.

In various exemplary embodiments of the invention the method is used for predicting liver cirrhosis.

The method begins at 10 and continues to 11 at which a plurality of parameters characterizing a subject is obtained. The inventors discovered that the probability developing the liver associated disease can be predicted for subjects that have not been diagnosed previously as being at risk for developing the liver associated disease (e.g., healthy subjects). Thus, in some embodiments of the present invention the subject is a healthy subject, and in some embodiments of the present invention the subject is not healthy but has not been diagnosed previously as being at risk for developing the liver associated disease.

Embodiments in which the subject is diagnosed as being at risk of developing liver associated disease are also contemplated.

As used herein a subject diagnosed as being at risk of developing liver associated disease is defined as a subject diagnosed with one or more of: VIRAL HEPATITIS B WITHOUT HEPATIC COMA & HEPATITIS DELTA-92, CHR. HEPATITIS C WITHOUT HEPATIC COMA, MALIGNANT NEOPLASM OF LIVER, PRIMARY, LIPIDOSES, ALPHA-1-ANTITRYPSIN DEFICIENCY, OTHER HEMOCHROMATOSIS, DISORDERS OF COPPER METABOLISM, ALCOHOL ABUSE, UNSPECIFIED DRINKING BEHAVIOR, BUDD-CHIARI SYNDROME, ALCOHOLIC FATTY LIVER, AUTOIMMUNE HEPATITIS, BILIARY CIRRHOSIS, CHOLANGITIS, HEPATITIS B CARRIER, HEPATITIS C CARRIER, and LIVER REPLACED BY TRANSPLANT. A subject not at risk of developing liver associated disease is defined as a subject that has not been diagnosed with any of the above conditions.

At least one of the parameters that are obtained at 11, more preferably more than one of these parameters, more preferably at least 2 or at least 3 or at least 4 or at least 5 or at least 6 or at least 7 or at least 8 or at least 9 or at least 10 of the parameters are extracted from an electronic health record associated with the subject. Parameters extracted from an electronic health record can include, but are not limited to, age, anthropometric parameters (e.g., height, weight, body mass index), blood pressure measurements, blood and urine laboratory tests, diagnoses recorded by physicians, and/or pharmaceuticals prescribed to the subject.

A list of parameters from which the parameters can be selected is provided in Table 10 of the Examples section that follows. In some embodiments of the present invention at least one or at least two or at least three or at least four or at least five or at least six or at least seven or at least eight or at least nine or at least ten of the parameters are selected from the parameters listed in Table 10. Preferably, but not necessarily, at least one or at least one or at least two or at least three of the parameters are selected from the parameters that are listed at lines 1-5 more preferably lines 1-4 more preferably lines 1-3 of Table 10.

Also contemplated are embodiments in the parameters include, preferably in addition to the above parameters, one or more of: anthropometric parameters (e.g., height, weight, body mass index), a parameter indicative of the age of the subject, one or more parameters indicative of previous diagnoses related to the subject or a family member thereof.

In some embodiments of the present invention the parameters include only parameters extracted from an electronic health record associated with the subject.

The number of parameters that are extracted from an electronic health record associated with the subject is preferably at least 5 or at least 6 or at least 7 or at least 8 or at least 9 or at least 10.

In some embodiments of the present invention at least one of the parameters is extracted from a body liquid test applied to the subject. In some embodiments of the present invention the parameters comprise a blood level of aspartate aminotransferase and a platelet score.

In some embodiments of the present invention the parameters are devoid of blood hyaluronic acid concentration, and blood procollagen III-NP concentration.

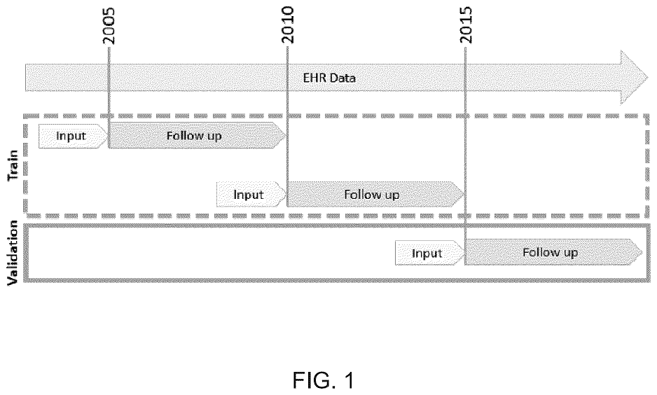

Referring again to FIG. 1, the method proceeds to 12 at which a computer readable medium storing a machine learning procedure is accessed. The machine learning procedure is trained for predicting probabilities for liver associated disease.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

Following is an overview of some machine learning procedures suitable for the present embodiments.

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

In KNN analysis, the affinity or closeness of objects is determined. The affinity is also known as distance in a feature space between objects. Based on the determined distances, the objects are clustered and an outlier is detected. Thus, the KNN analysis is a technique to find distance-based outliers based on the distance of an object from its kth-nearest neighbors in the feature space. Specifically, each object is ranked on the basis of its distance to its kth-nearest neighbors. The farthest away object is declared the outlier. In some cases the farthest objects are declared outliers. That is, an object is an outlier with respect to parameters, such as, a k number of neighbors and a specified distance, if no more than k objects are at the specified distance or less from the object. The KNN analysis is a classification technique that uses supervised learning. An item is presented and compared to a training set with two or more classes. The item is assigned to the class that is most common amongst its k-nearest neighbors. That is, compute the distance to all the items in the training set to find the k nearest, and extract the majority class from the k and assign to item.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within a dataset. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in explaining the response to the treatment. Symmetrical uncertainty is an algorithm that can be used by a feature selection algorithm, according to some embodiments of the present invention. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on predicting probability for developing a liver associated disease, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the dataset or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular parameter influences on the probability for developing a liver associated disease) or a value (e.g., the predicted probability for developing a liver associated disease). The leaf nodes can also contain additional information about the represented classification such as a confidence score that measures a confidence level in the represented classification (i.e., the accuracy of the prediction).

Regression techniques which may be used in accordance with some embodiments the present invention include, but are not limited to linear Regression, Multiple Regression, logistic regression, probit regression, ordinal logistic regression ordinal Probit-Regression, Poisson Regression, negative binomial Regression, multinomial logistic Regression (MLR) and truncated regression.

A logistic regression or logit regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regression may also predict the probability of occurrence for each data point. Logistic regressions also include a multinomial variant. The multinomial logistic regression model is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.). For binary-valued variables, a cutoff between the 0 and 1 associations is typically determined using the Yuden Index.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the probability for developing a liver associated disease. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based techniques generate a new model for each instance, instead of basing predictions on trees or networks generated (once) from a training set.

The term “instance”, in the context of machine learning, refers to an example from a dataset.

Instance-based techniques typically store the entire dataset in memory and build a model from a set of records similar to those being tested. This similarity can be evaluated, for example, through nearest-neighbor or locally weighted methods, e.g., using Euclidian distances. Once a set of records is selected, the final model may be built using several different techniques, such as the naive Bayes.

Neural networks are a class of algorithms based on a concept of inter-connected “neurons.” In a typical neural network, neurons contain data values, each of which affects the value of a connected neuron according to connections with pre-defined strengths, and whether the sum of connections to each particular neuron meets a pre-defined threshold. By determining proper connection strengths and threshold values (a process also referred to as training), a neural network can achieve efficient recognition of images and characters. Oftentimes, these neurons are grouped into layers in order to make connections between groups more obvious and to each computation of values. Each layer of the network may have differing numbers of neurons, and these may or may not be related to particular qualities of the input data.

In one implementation, called a fully-connected neural network, each of the neurons in a particular layer is connected to and provides input value to those in the next layer. These input values are then summed and this sum compared to a bias, or threshold. If the value exceeds the threshold for a particular neuron, that neuron then holds a positive value which can be used as input to neurons in the next layer of neurons. This computation continues through the various layers of the neural network, until it reaches a final layer. At this point, the output of the neural network routine can be read from the values in the final layer. Unlike fully-connected neural networks, convolutional neural networks operate by associating an array of values with each neuron, rather than a single value. The transformation of a neuron value for the subsequent layer is generalized from multiplication to convolution.

The machine learning procedure used according to some embodiments of the present invention is a trained machine learning procedure, which provides output that is related non-linearly to the parameters with which it is fed.

A machine learning procedure can be trained according to some embodiments of the present invention by feeding a machine learning training program with parameters that characterizes each of a cohort of subjects that has been diagnosed as either having or not having a liver associated disease. Once the data are fed, the machine learning training program generates a trained machine learning procedure which can then be used without the need to re-train it.

For example, when it is desired to employ decision trees, machine learning training program learns the structure of each tree in a plurality of decision trees (e.g., how many nodes there are in each tree, and how these are connected to one another), and also selects the decision rules for split nodes of each tree. At least a portion of the decision rules relate to one or more of the parameters that characterize the subject. A simple decision rule may be a threshold for the value of a particular parameter, but more complex rules, relating to more than one parameters are also contemplated. The machine learning training program also accumulates data at the leaves of the trees. The structures of the trees, the decision rules for the split nodes, and the data at the leaves are all selected by the machine learning training program, automatically and typically without user intervention, such that the parameters at the root of the trees provide the probability for developing a liver associated disease at the leaves of the trees. The final result of the machine learning training program in this case is a set of trees, where the structures, the decision rules for split nodes, and leaf data for each trees are defined by the machine learning training program.

The method proceeds to 13 at which the trained machine learning procedure is fed with the parameters, and to 14 at which an output indicative of the probability that he subject has, or expected to develop, a liver associated disease, is received from the procedure.

In some embodiments of the present invention the method proceeds to 15 at which an output indicative of an expected onset time of the liver-associated disease in the subject is received from the procedure. This is advantageous because it provides the method with the ability to predict how much time is expected to pass between the time at which the parameters were obtained from the subject and the time at which the disease diagnosis is expected. The expected onset time can be provided as a continuous score expressed, for example, in units of time (e.g., days, months, years). An output indicative of an expected onset time can be ensured by a judicious selection of the training process and the machine learning procedure. Preferably, the machine learning procedure is a procedure that is constructed based on a survival analysis model and that is trained on data that include both censored and uncensored events.

In some embodiments of the present invention the method proceeds to 16 at which, a predetermined time ahead of the expected onset time, the subject is enrolled to a periodic monitoring for the onset of the liver associated disease. Preferably, the subject is enrolled to the periodic monitoring M months ahead of the expected onset time, where M is from about 12 to about 60. Preferred period for monitoring the enrolled subject is every D days, where D is from about 7 to about 21. The monitoring can be by any technique known in the art for diagnosing a liver associated disease. Representative examples of such technologies include, without limitation, biopsy, MRI elastography, transient elastography, ultrasound elastography, and a fibrosis test.

In some embodiments of the present invention the method proceeds to 17 at which a report predating to the probability is generated. The report can be displayed on a display device or transmitted to a computer readable medium.

In some embodiments of the present invention the method proceeds to 18 at which measures for reducing the probability or preventing development of the disease are applied. The measures can be in the form of administering of prescribing a pharmaceutical agent that reduces the probability of developing the liver disease. Representative examples of such agents suitable for the present embodiments include, without limitation, the pharmaceutical agent disclosed in U.S. Published application No. 20210015818, U.S. Pat. No. 10,624,917 and European Patent No. 3 020 405, the contents of which are hereby incorporated by reference. Alternatively, or additionally, the measures can include recommendations for a change of life style, and/or prescription of nutritional supplements or a dietary formulation or other dietary changes.

The method ends at 19.

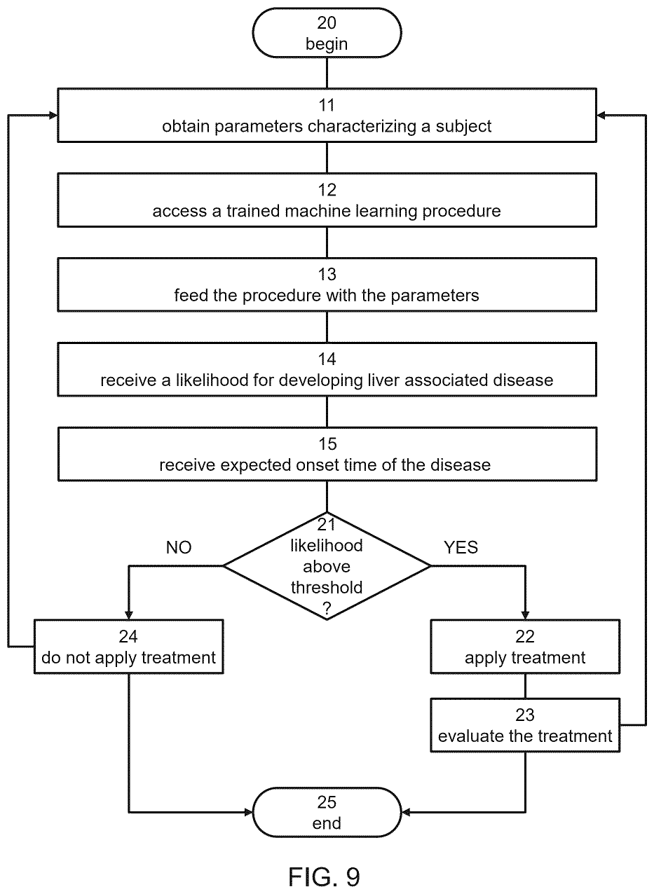

FIG. 9 is a flowchart diagram of a method suitable for evaluating efficacy of a treatment for a liver associated disease, according to some embodiments of the present invention. The method can be used to test whether a specific dosage of a pharmaceutical agent that has a known effect on the liver associated disease is sufficient for treating the disease, and/or to evaluate the efficacy of a new formulation containing a pharmaceutical agent that has a known effect on the liver associated disease, and/or evaluate whether a new pharmaceutical agent is effective for treating the liver associated disease. The method can also be used to test a treatment that includes non-pharmaceutical therapy procedures. The method is particularly useful for enrolment of subjects into a research group to participate in a research directed to evaluate the efficacy of the treatment.

The method begins at 20 and continues to operations 11, 12, 13 and 14 as further detailed hereinabove. In some embodiments of the present invention the method also proceed to 15 as further detailed hereinabove. Once the probability, and optionally the expected onset, is/are obtained, the method continues to decision 21 at which the probability is compared to a predetermined threshold. The subject can be enrolled into the research group if the probability is above the threshold, and not enrolled to the research group otherwise.

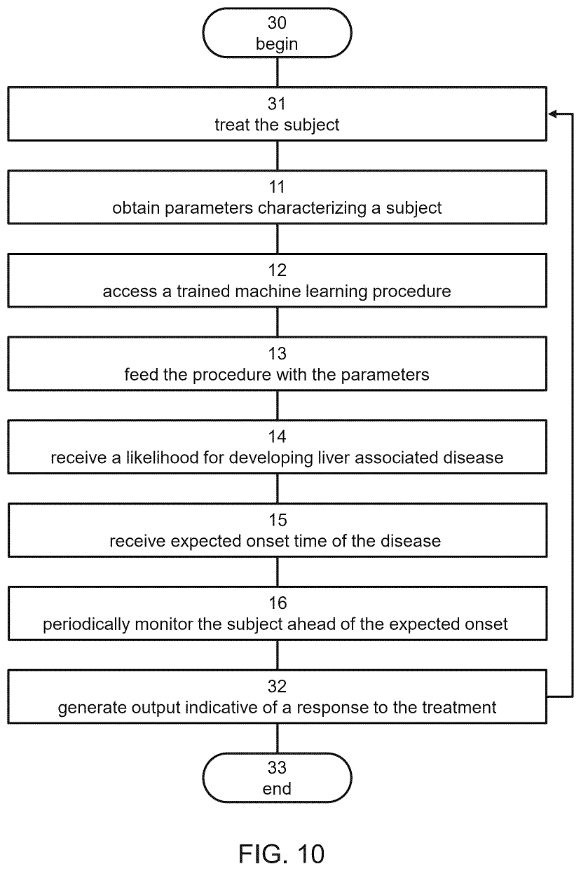

In cases in which the probability is above the threshold, the method proceeds to 22 at which the treatment is applied to the subject, and to 23 at which the efficacy of the treatment is evaluated, for example, by executing one or more of the techniques described above with respect to operation 16 of method 10. Also contemplated are embodiments in which operation 23 includes executing selected operations of method 30 described below with reference to FIG. 10. From 23 the method can proceed to 26 at which it ends, or loop back to 11, for repeating the execution for another subject. If the probability is not above the threshold, no treatment is applied (24) to the subject, and the method can proceed to 26 or loop back to 11.

FIG. 10 is a flowchart diagram of a method suitable for predicting a response to a treatment-of-interest, according to some embodiments of the present invention. Typically, the treatment-of-interest is a treatment for a liver associated disease. The method can be used to predict the response of a particular subject to a specific dosage of a pharmaceutical agent that has a known effect on the liver associated disease, and/or to predict the response of a particular subject to a new formulation containing a pharmaceutical agent that has a known effect on the liver associated disease, and/or to predict the response of a particular subject a new pharmaceutical agent. The method can also be used to predict the response of a particular subject to a treatment that includes non-pharmaceutical therapy procedures. Pharmaceutical agents having a known effect on liver associated disease include, but are not limited to, antiviral agents, antibiotics and chelating agents.

The method begins at 30 and continues to 31 at which the subject is treated by the treatment-of-interest. The method continues to operations 11, 12, 13 and 14 as further detailed hereinabove. In some embodiments of the present invention the method also proceed to 15 as further detailed hereinabove. Once the probability, and optionally the expected onset, is/are obtained, the method continues to 32 at which an output indicative of the response of the subject to the treatment is generated based on the probability. The method can proceed to end 33 or loop back to 31 for repeating the execution for another subject.

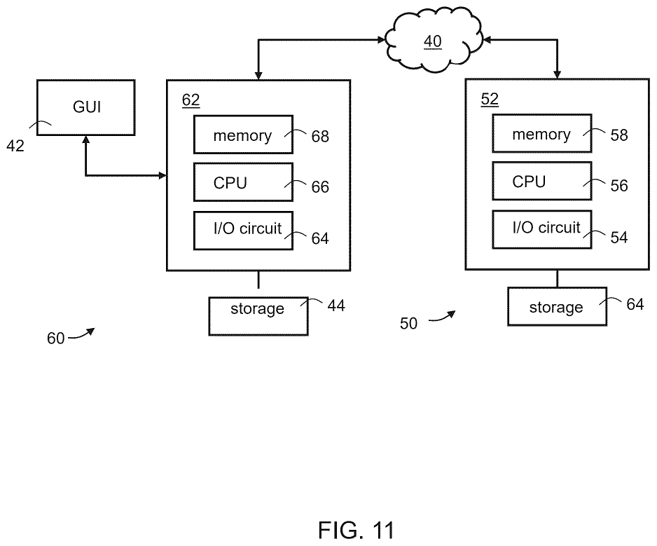

FIG. 11 illustrates a computing platform having a client computer 60 and a server computer 50. Client computer 60 has a hardware processor 62, which typically comprises an input/output (I/O) circuit 64, a hardware central processing unit (CPU) 66 (e.g., a hardware microprocessor), and a hardware memory 68 which typically includes both volatile memory and non-volatile memory. CPU 66 is in communication with I/O circuit 64 and memory 68. Client computer 60 preferably comprises a user interface, e.g., a graphical user interface (GUI), 42 in communication with processor 62. I/O circuit 64 preferably communicates information in appropriately structured form to and from GUI 42. Server computer 50 can similarly include a hardware processor 52, an I/O circuit 54, a hardware CPU 56, a hardware memory 58. I/O circuits 64 and 54 of client 60 and server 50 computers preferable operate as transceivers that communicate information with each other via a wired or wireless communication. For example, client 60 and server 50 computers can communicate via a network 40, such as a local area network (LAN), a wide area network (WAN) or the Internet. Server computer 50 can be in some embodiments be a part of a cloud computing resource of a cloud computing facility in communication with client computer 60 over the network 40.

GUI 42 and processor 62 can be integrated together within the same housing or they can be separate units communicating with each other. GUI 42 can optionally and preferably be part of a system including a dedicated CPU and I/O circuits (not shown) to allow GUI 42 to communicate with processor 62. Processor 62 issues to GUI 42 graphical and textual output generated by CPU 66. Processor 62 also receives from GUI 42 signals pertaining to control commands generated by GUI 42 in response to user input. GUI 42 can be of any type known in the art, such as, but not limited to, a keyboard and a display, a touch screen, and the like. In some embodiments, GUI 42 is a GUI of a mobile device such as a smartphone, a tablet, a smartwatch and the like. When GUI 42 is a GUI of a mobile device, the CPU circuit of the mobile device can serve as processor 62 and can execute the method optionally and preferably by executing code instructions.

Client 60 and server 50 computers can further comprise one or more computer-readable storage media 44, 64, respectively. Media 44 and 64 are preferably non-transitory storage media storing computer code instructions for executing the method of the present embodiments, and processors 62 and 52 execute these code instructions. The code instructions can be run by loading the respective code instructions into the respective execution memories 68 and 58 of the respective processors 62 and 52.

In operation, processor 62 of client computer 60 receives parameters characterizing a subject as further detailed hereinabove. Processor 62 can transmit the parameters to server computer 50 over network 40. Media 64 can store a machine learning procedure trained for predicting probabilities for developing a liver associated disease. Server computer 50 can access media 64, feed the stored procedure with the parameters received from client computer 60, and receive from the procedure an output indicative of the probability that the subject that is characterized by the parameters has, or is expected to develop, a liver associated disease. Server computer 50 can also transmit to client computer 60 the obtained probability, and client computer 60 can display this information on GUI 42. Alternatively, media 44 can store the machine learning procedure, in which case server computer 60 accesses media 44, feeds the stored procedure with the parameters, receives from the procedure an output indicative of the probability that the subject has, or is expected to develop, a liver associated disease, and displays this information on GUI 42. In this case, in is not necessary for the computing platform to include server computer 50.

Also contemplated are embodiments in which media 64 store the machine learning procedure and in which server computer 50 can access media 64 and transmits the stored procedure to client computer 60. Server computer 60 feeds the procedure received from server 50 with the parameters, receives from the procedure an output indicative of the probability that the subject has, or is expected to develop, a liver associated disease, and displays this information on GUI 42.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Liver diseases account for approximately two-million annual deaths worldwide, half due to liver cirrhosis complications. Diagnostic tests are performed mainly in high-risk populations, rendering most patients undiagnosed for several years. The Inventors appreciate that available non-invasive tests suffer from low accuracy making them suboptimal for screening in the general population. This Example describes an accurate methodology is needed to direct high-risk individuals for clinical follow-ups, using nationwide electronic health records (EHR).

Cirrhosis, the third leading cause of death in people aged 45-64 and the 11th most common cause of death worldwide [2], is associated with morbidity from liver disease [3,4]. Chronic liver inflammation followed by hepatic fibrosis can lead to liver cirrhosis, an irreversible condition. Treatments include lifestyle modification (alcohol abstinence and weight loss), screening for Hepatocellular carcinoma, and avoiding the use of hepatotoxic drugs such as antiviral treatments.

Most patients with liver cirrhosis are not diagnosed at early stages of disease, while in fact 75% of them present with decompensated cirrhosis as the first manifestation [5]. Decompensation is defined by the development of ascites, variceal bleeding or encephalopathy, and occurs at a rate of up to 11% compensated cirrhotic patients per year [6]. Liver fibrosis is classified into four stages: no fibrosis—stage 1, with increasing severity of the fibrosis to stage 4 which represent cirrhosis.

The degree of fibrosis is determined by liver biopsy. The Inventors appreciate that liver biopsy is invasive, costly, and can lead to grave complications. Biopsy is therefore mainly used to determine the etiology of liver disease, with wider use of non-invasive tools for determining fibrosis staging. Transient Elastography (TE) measures the stiffness of the liver tissue in kilopascals (kPa) using pulse-echo ultrasound acquisitions, which has been found to correlate well with the fibrosis stage [7]. MRI elastography is superior to TE in assessing fibrosis but is costly.

TE was found to have a sensitivity of 86% and a specificity of 97% in a population known to have 6% advanced fibrosis [9]. Patient referrals to TE are reaching mostly individuals who are already acknowledged as at-risk for liver disease (e.g. due to alcohol abuse or hepatitis), mainly due to reduced accessibility and availability of TE in primary care.

The Inventors therefore appreciate that timely diagnosis in the majority of patients is not achieved.

Several low cost blood tests-based risk scores have been developed and are commonly used. These include Fibrosis-4 test (FIB-4) and the non-alcoholic fatty liver disease fibrosis score [10,11]. The Inventors found that these tests of liver cirrhosis suffer from low accuracy, and misclassify a third of the patients in the general population [12]. Other population-based studies using non-invasive tests demonstrated that 18%-27% of at-risk populations have undetected liver fibrosis and established cirrhosis [13].

This Example describes a model for risk assessment in the general population for liver cirrhosis that is based on longitudinal nationwide EHR data. The model's performance was evaluated on true cases from the community and the findings were validated in the clinical setting by performing TE and blood tests.

### Methods

### Electronic Data

EHR data were obtained from Clalit, Israel's largest healthcare provider, containing more than 5.5 million demographically heterogeneous members dating back to 2002 [20]. Anonymized medical records comprise members' full clinical registry including lab test results and diagnoses recorded as International Classification of Diseases, Ninth Edition (ICD9) codes [21]. Electronic study protocol was approved by the Clalit Helsinki committee 0195-17-COM2. Since this study is based on retrospective data, it was exempt from the provision of patients' written informed consent.

### Retrospective Study Design

EHR data was split temporarily using a rolling-origin-update methodology [22]. FIG. 1 is an illustration showing an overview of the retrospective cohort. In a first, retrospective stage of the study, EHR data was split into three non-overlapping time periods: 2005-2010, 2010-2015 and 2015-2020. Each time period was uniquely defined by an index-date (T0), marked in FIG. 1 as a gray vertical line. Each time period includes input lab test results from the previous year (marked in yellow), and a follow-up period of up to five years marked in pink. The earlier two time periods were used to train the model dashed gray box, and the last period was used for validation gray box.

Since a member could be recorded during more than one time period, a sample was defined as a pair of a member and an index-date. For each time period a cohort, an input data, and follow-up outcomes were constructed. Table 1, below summarizes the study protocol per cohort.

TABLE 1

| Protocol | Retrospective electronic cohort | Prospective clinical cohort |
|---|---|---|
| Eligibility | Age at index-date is between 40 to 75. | Age at index-date is between 40 to 75. |
| | Enrollment in Clalit at index-date. | Enrollment in Clalit's Afula subdistrict. |
| | Available blood test values for HB, PLT and WBC from | Available blood test values for HB, PLT |
| | the year prior to index-date. | and WBC. |
| Exclusion | Liver cirrhosis diagnosis | Liver cirrhosis diagnosis |
| criteria | (see ICD9 codes in Table 2 below) prior to index-date. | (see ICD9 codes in Table 2 below). |
| | Diagnosis of exclusion diagnoses | Diagnosis of exclusion diagnoses |
| | (see ICD9 codes in Table 2 below) prior to index-date. | (see ICD9 codes in Table 2 below). |
| Follow-up | Study start is at one of three index-dates: | Study start is on Dec. 8, 2021. |
| | Jan. 1, 2005 | Individuals who were only contacted were |
| | Jan. 1, 2010 | followed up until contact. |
| | Jan. 1, 2015 | Individuals who arrived at the clinic were |
| | Follow-up starts at index-date and ends at the earliest of: | followed up until the date of hepatology |
| | Liver diagnosis event | consultation. |
| | Exclusion diagnosis event | |
| | Loss to follow-up (due to either death or leaving Clalit) | |
| | End of study period (five years after index-date) | |
| Outcomes | Liver diagnosis (see ICD9 codes in Table 2 below) | For individuals only contacted: mortality |
| | | For individuals who underwent hepatology |
| | | consultation: |
| | | Liver stiffness (kPa) |
| | | Steatosis grade (CAP score) |
| | | Weight |
| | | Height |
| | | WHO Alcohol Use |
| | | Disorders Identification Test (AUDIT) |
| | | questionnaire score |

TABLE 2

| Exclusion | |
|---|---|
| ICD9 code | Diagnosis description |
| 7030 | VIRAL HEPATITIS B WITHOUT HEPATIC COMA & |
| | HEPATITIS DELTA −92 |
| 7054 | CHR. HEPATITIS C WITHOUT HEPATIC COMA |
| 1550 | MALIGNANT NEOPLASM OF LIVER, PRIMARY |
| 2727 | LIPIDOSES |
| 2734 | ALPHA-1-ANTITRYPSIN DEFICIENCY |
| 27503 | OTHER HEMOCHROMATOSIS |
| 2751 | DISORDERS OF COPPER METABOLISM |
| 30500 | ALCOHOL ABUSE, UNSPECIFIED DRINKING |
| | BEHAVIOR |
| 4530 | BUDD-CHIARI SYNDROME |
| 5710 | ALCOHOLIC FATTY LIVER |
| 57142 | AUTOIMMUNE HEPATITIS |
| 5716 | BILIARY CIRRHOSIS |
| 5761 | CHOLANGITIS |
| V0261 | HEPATITIS B CARRIER |
| V0262 | HEPATITIS C CARRIER |
| V427 | LIVER REPLACED BY TRANSPLANT |

Eligibility was determined per index-date, including only members 40-70 years old, who were Clalit members at index-date. Only members with valid lab test results of hemoglobin (HB), platelet (PLT) and white blood count (WBC) from the year before index-date were considered. Records with prior liver cirrhosis or one of predefined exclusion diagnoses (see ICD9 codes in Table 2), where excluded. Table 3, below, summarizes the inclusion-exclusion criteria.

TABLE 3

| Inclusion | Exclusion |
|---|---|
| Ages 40-70 | Prior liver cirrhosis diagnosis |
| Existing values of HB, PLT and | Prior exclusion diagnosis |
| WBC (in the electronic cohort lab | |
| tests were only considered up to a | |
| year prior to index-date) | |
| Clalit healthcare members | |

For each time period input data was defined as sex and age at index-date, as well as latest lab test results: HB, PLT, WBC, Aspartate Aminotransferase (AST), Alanine Transaminase (ALT), Albumin, Bilirubin, Prothrombin Time International Normalized Ratio (PT-INR), Vitamin B12, Glucose, Hemoglobin A1c, Cholesterol, HDL cholesterol, LDL cholesterol, triglycerides and total protein. Only latest lab test results within predefined thresholds taken during the one-year prior to index-date were considered. Table 4, below, summarizes the predefined thresholds. The thresholds provide upper and lower bounds for valid lab test results. In Table 4, an empty cell indicates that no upper bound was used for the respective lab test result. Some of these values are beyond normal distribution of healthy lab test results. This was done under the assumption that individuals who suffer from liver cirrhosis and are undiagnosed might have abnormal lab test results.

TABLE 4

| Name of lab test | Minimal value | Maximal Value |
|---|---|---|
| HB | 2 | 20 |
| PLT | 10 | 1000 |
| WBC | 1 | 20 |
| AST (GOT) | 3 | |
| ALT (GPT) | 3 | |
| ALBUMIN | 1 | 6 |
| BILIRUBIN TOTAL | 0.1 | 100 |
| PT-INR | 0.7 | 6 |
| VITAMIN B12 | 5 | 1500 |
| GLUCOSE | 5 | 1000 |
| HEMOGLOBIN A1C % | 0 | 20 |
| CHOLESTEROL | 10 | |
| CHOLESTEROL - HDL | 10 | 100 |
| CHOLESTEROL - LDL calc | 10 | |
| TRIGLYCERIDES | 10 | |
| PROTEIN - TOTAL | 4 | 10 |

In order to avoid overlap between follow-up periods, e.g., to reduce information leakage, follow-up was limited to five years post index-date. Individual follow-up ended at an observed outcome event of liver cirrhosis diagnosis (see liver cirrhosis diagnosis ICD-9 codes in Table 5, below), or at a right censoring event, where no liver cirrhosis diagnosis occurred but can also no longer be observed. Right censoring events were: exclusion diagnosis (Table 2), death, disenrollment from Clalit, or the end of the five-year follow-up period, whichever occurred first.

TABLE 5

| Diagnosis | |
|---|---|
| ICD-9 code | Diagnosis description |
| 1550 | MALIGNANT NEOPLASM OF LIVER, PRIMARY |
| 452 | PORTAL VEIN THROMBOSIS |
| 4560 | ESOPHAGEAL VARICES WITH BLEEDING |
| 4561 | ESOPHAGEAL VARICES WITHOUT MENTION OF |
| | BLEEDING |
| 4562 | ESOPHAGEAL VARICES IN DISEASES CLASSIFIED |
| | ELSEWHERE |
| 45620 | ESOPHAGEAL VARICES IN DISEASES |
| | CLASS. ELSEWHERE, WITH BLEEDING |
| 45621 | ESOPHAGEAL VARICES IN |
| | DIS. CLASSIF. ELSEWHERE, WITHOUT BLEEDING |
| 56723 | SPONTANEOUS BACTERIAL PERITONITIS |
| 571 | CHRONIC LIVER DISEASE AND CIRRHOSIS |
| 5712 | ALCOHOLIC CIRRHOSIS OF LIVER |
| 5715 | CIRRHOSIS OF LIVER WITHOUT MENTION OF |
| | ALCOHOL |
| 5722 | HEPATIC COMA |
| 5724 | HEPATORENAL SYNDROME |
| 7895 | ASCITES |
| 78959 | OTHER ASCITES |
| D97 | CIRRHOSIS/OTHER LIVER DISEASE |
| Z4291 | INJECTION OR LIGATION OF ESOPHAGEAL |
| | VARICES |

### Model Training and Evaluation

A machine-learning model was constructed to predict the hazard for liver cirrhosis diagnosis based on the input data described above. A survival analysis gradient boosting decision trees regression model was trained on the earlier two time periods and its predictions was evaluated on the latest period (using XGBoost [23] with objective=“survival:cox”, n_estimators=100 and base_score=1). Survival analysis models allow prediction of time-to-event of first diagnosis rather than whether a diagnosis will occur within the follow-up period, making them a better fit for prioritization of individuals in general populations. Furthermore, unlike linear regression models, gradient boosting decision trees enable maintaining missing data without the need for imputation as well as account for higher-order nonlinear interactions between variables.

Prediction outcomes were evaluated using c-index and accuracy measures. All measures were compared between the train and validation periods to analyze performance on unseen data, as well as to alternative FIB-4 scoring.

Following retrospective evaluations, a new model was trained with the same parameters on all three EHR time periods, for prediction of prospective cohort.

### Prospective Clinical Cohort

External prospective validation was performed on a clinical cohort from Clalit's Afula subdistrict.

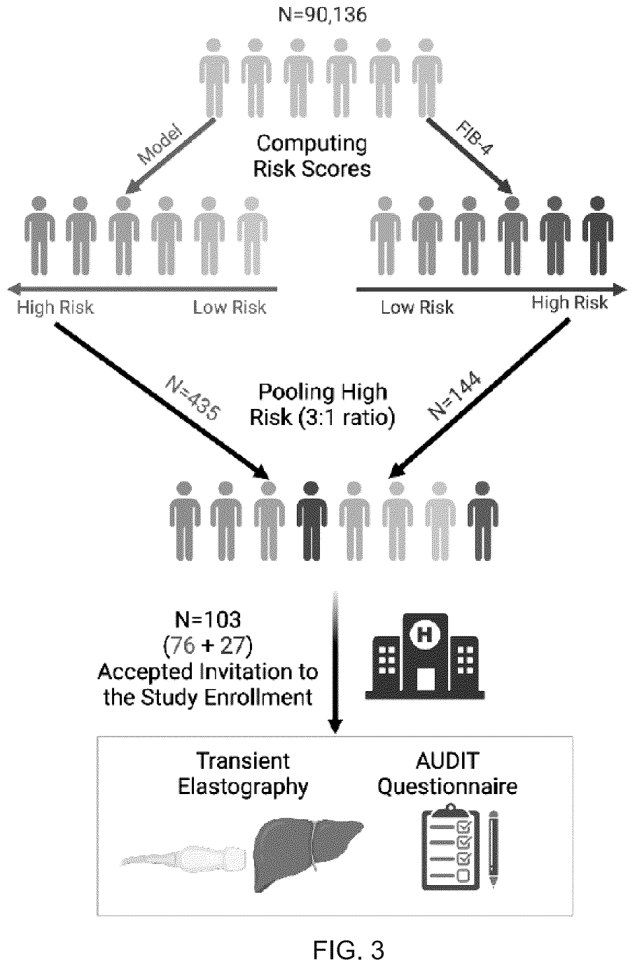

FIG. 3 is an illustration showing an overview of the prospective cohort. Latest age, gender and laboratory test results were obtained from 115,211 Afula subdistrict's Clalit members, 90,136 of which passed inclusion exclusion criteria. Model predicted risks and FIB-4 risks were computed to all individuals, identifying low to high risk individuals for each risk form (high risk displayed as darker and low risk as lighter). A predicted risk via the EHR-based model was computed for all individuals, as well as FIB-4 scores when possible (meaning AST and ALT levels were available). For each risk score methodology, individuals were ranked from highest risk to lowest. Highest risk individuals of the two lists were pooled with a 3:1 ratio of model to FIB-4, and were not overlapping between the two methodologies, meaning each individual's origin was from a single risk score. To ensure a double blinded study, computed risk scores were concealed during the clinical trial. All individuals were contacted and invited for hepatology consultation and noninvasive fibrosis tests, and cases of morbidity were recorded. Those (n=103) who arrived at the clinic underwent TE exams, height and weight measurements, and filled out the AUDIT questionnaire. Liver stiffness was measured in kPa and steatosis grade measured via CAP score. The clinical cohort alongside the entire study protocol was approved by Emek Medical Center Helsinki Committee 0208-20-EMC.

### Results

### Retrospective Cohort—Demographic Characteristics

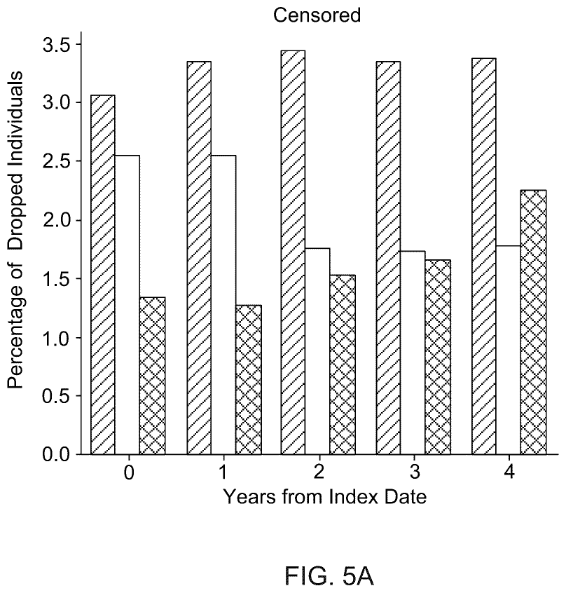

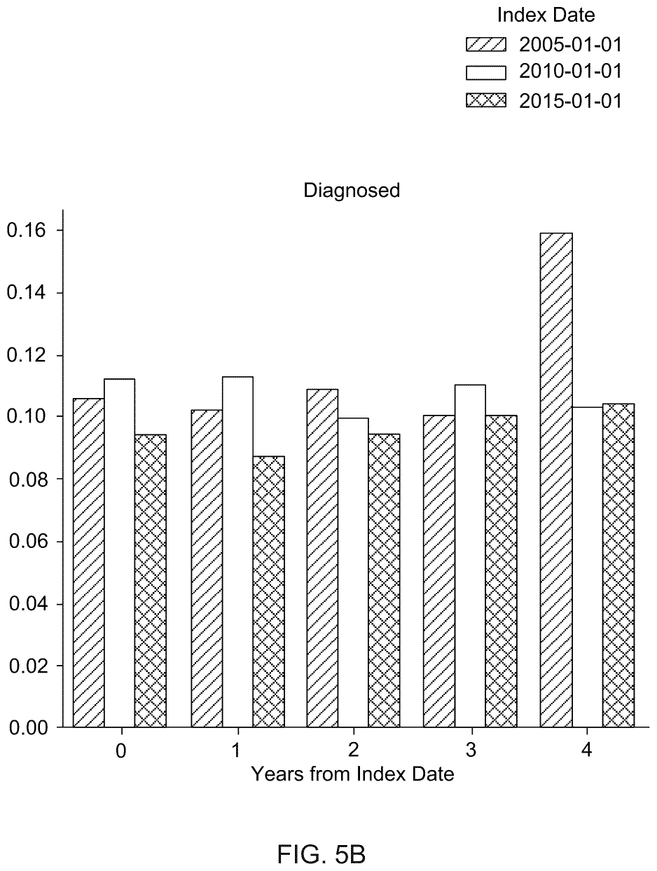

Study period included electronic EHR data from Clalit Health Services (Clalit) spanning from 2004 to 2020, and was divided into three distinct non-overlapping periods (see Methods, FIG. 1). Each period was uniquely defined by an index-date (TO) and a subsequent five-year follow-up. A total of 2,255,580 samples were distinguished (a sample is defined as an eligible individual at an index-date) who passed inclusion exclusion criteria. Of those, the mean age was 56.9 (SD=9.9), and 57.5% were female (Tables 6 and 7, below). A total of 11,337 individuals were diagnosed with liver cirrhosis during their five-year follow-ups (0.5%). FIGS. 5A and 5B show percentage of individuals who halted follow-up per year post index-date. Shown are the annual rates of right censoring (FIG. 5A) and of liver cirrhosis diagnosis (FIG. 5B) per year from the start of follow-up. Colors indicate the different temporal validation periods.

TABLE 6

| | | Retrospective | | Prospective | |
|---|---|---|---|---|---|
| | | electronic | | clinical | |
| Group | Characteristic | cohort | | cohort | |
| Demographics | N | 2,255,580 | | 90,136 | |
| | Age, mean | 56.9 | (9.9) | 56.9 | (9.9) |
| | (SD), y | | | | |
| | Sex, N (%), | 1,296,695 | (57.5) | 49,782 | (55.2) |
| | Female | | | | |
| | Sex, N (%), | 958,885 | (42.5) | 40,354 | (44.8) |
| | Male | | | | |
| Laboratory test | HB | 13.6 | (1.5) | 13.6 | (1.6) |
| results, mean (SD) | PLT | 251.9 | (68.2) | 254.7 | (70.5) |
| | WBC | 7.2 | (2.1) | 7.3 | (2.6) |
| | AST (GOT) | 22.0 | (11.8) | 22.1 | (18.0) |
| | ALT (GPT) | 21.8 | (16.3) | 21.9 | (15.5) |
| | ALBUMIN | 4.3 | (0.3) | 4.1 | (0.3) |
| | BILIRUBIN | 0.6 | (0.3) | 0.6 | (0.3) |
| | TOTAL | | | | |
| | PT-INR | 1.1 | (0.5) | 1.0 | (0.3) |
| | VITAMIN B12 | 322.0 | (155.2) | 950.7 | (463.5) |
| | GLUCOSE | 104.6 | (36.3) | 107.6 | (36.5) |
| | HEMOGLOBIN | 6.8 | (1.6) | 6.2 | (1.3) |
| | A1C % | | | | |
| | CHOLESTEROL | 192.0 | (38.6) | 181.6 | (40.8) |
| | CHOLESTEROL- | 49.6 | (12.9) | 50.1 | (12.6) |
| | HDL | | | | |
| | CHOLESTEROL- | 114.1 | (33.4) | 105.4 | (33.7) |
| | LDL calc | | | | |
| | TRIGLYCERIDES | 141.7 | (87.4) | 134.6 | (87.0) |
| | PROTEIN- | 7.3 | (0.5) | 29.9 | (41.7) |
| | TOTAL | | | | |
| FIB-4 Score, N (%) | <1.45 | 1,620,673 | (71.9) | 71,722 | (79.6) |
| | 1.45-3.25 | 383,035 | (17.0) | 15,688 | (17.4) |
| | >3.25 | 10,679 | (0.5) | 447 | (0.5) |
| | Missing | 241,193 | (10.7) | 2,279 | (2.5) |

| | | | | |
|---|---|---|---|---|
| Physical health | DIABETES | 107,634 | (8.3) | NA |
| conditions*, N (%) | MELLITUS | | | |
| | (250) | | | |
| | ESSENTIAL | 193,331 | (15.0) | NA |

HYPERTENSION
(401)

*Physical health conditions include the number of samples who had a prior diagnosis (ICD9 marked in brackets). The percent of diagnosed patients was less than the general population (10.6% for diabetes mellitus, and 17.5% for essential hypertension). Diagnosis data was unavailable for the prospective cohort

TABLE 7

| | | electronic | | electronic | | electronic | | prospective | |
|---|---|---|---|---|---|---|---|---|---|
| | | cohort | | cohort | | cohort | | clinical | |
| Group | Characteristic | 2005 Jan. 1 | | 2010 Jan. 1 | | 2015 Jan. 1 | | cohort | |
| Demographics | N | 678,512 | | 760,587 | | 816,481 | | 90,136 | |
| | Age, mean | 57.0 | (10.0) | 56.8 | (9.7) | 56.8 | (9.9) | 56.9 | (9.9) |
| | (SD), y | | | | | | | | |
| | Sex, Female | 401,071 | (59.1) | 435,372 | (57.2) | 460,252 | (56.4) | 49,782 | (55.2) |
| | Sex, Male | 277,441 | (40.9) | 325,215 | (42.8) | 356,229 | (43.6) | 40,354 | (44.8) |
| Laboratory test | HB | 13.6 | (1.5) | 13.6 | (1.5) | 13.6 | (1.5) | 13.6 | (1.6) |
| results, mean | PLT | 250.9 | (67.8) | 257.9 | (69.6) | 247.3 | (66.9) | 254.7 | (70.5) |
| (SD) | WBC | 7.2 | (2.1) | 7.4 | (2.1) | 7.1 | (2.1) | 7.3 | (2.6) |
| | AST (GOT) | 20.7 | (12.2) | 22.5 | (12.5) | 22.7 | (10.6) | 22.1 | (18.0) |
| | ALT (GPT) | 21.8 | (16.9) | 21.8 | (17.1) | 21.9 | (14.9) | 21.9 | (15.5) |
| | ALBUMIN | 4.3 | (0.3) | 4.3 | (0.3) | 4.3 | (0.3) | 4.1 | (0.3) |
| | BILIRUBIN | 0.6 | (0.4) | 0.6 | (0.3) | 0.6 | (0.3) | 0.6 | (0.3) |
| | TOTAL | | | | | | | | |
| | PT-INR | 1.2 | (0.5) | 1.1 | (0.5) | 1.0 | (0.4) | 1.0 | (0.3) |
| | VITAMIN B12 | 310.3 | (152.0) | 317.1 | (151.3) | 333.8 | (160.0) | 950.7 | (463.5) |
| | GLUCOSE | 103.8 | (39.3) | 104.2 | (35.1) | 105.5 | (35.0) | 107.6 | (36.5) |
| | HEMOGLOBIN | 7.3 | (1.8) | 6.8 | (1.5) | 6.5 | (1.4) | 6.2 | (1.3) |
| | A1C % | | | | | | | | |
| | CHOLESTEROL | 201.6 | (38.5) | 189.7 | (37.5) | 186.6 | (38.4) | 181.6 | (40.8) |
| | CHOLESTEROL- | 49.7 | (12.8) | 49.4 | (12.9) | 49.8 | (13.0) | 50.1 | (12.6) |
| | HDL | | | | | | | | |
| | CHOLESTEROL- | 122.4 | (35.2) | 112.3 | (31.7) | 109.6 | (32.4) | 105.4 | (33.7) |
| | LDL calc | | | | | | | | |
| | TRIGLYCERIDES | 147.1 | (91.9) | 141.0 | (84.7) | 138.2 | (86.2) | 134.6 | (87.0) |
| | PROTEIN- | 7.3 | (0.5) | 7.4 | (0.5) | 7.1 | (0.4) | 29.9 | (41.7) |
| | TOTAL | | | | | | | | |
| FIB-4 Score | <1.45 | 480,335 | (70.8) | 558,080 | (73.4) | 582,258 | (71.3) | 71,722 | (79.6) |
| | 1.45-3.25 | 100,365 | (14.8) | 122,193 | (16.1) | 160,477 | (19.7) | 15,688 | (17.4) |
| | >3.25 | 3,463 | (0.5) | 3,276 | (0.4) | 3,940 | (0.5) | 447 | (0.5) |
| | Missing | 94,349 | (13.9) | 77,038 | (10.1) | 69,806 | (8.5) | 2,279 | (2.5) |

| | | | | | | | |
|---|---|---|---|---|---|---|---|
| Physical health | DIABETES | 88,704 | (13.1) | 144,672 | (19.0) | 172,501 | (21.1) |
| conditions | MELLITUS | | | | | | |
| | (250) | | | | | | |
| | ESSENTIAL | 159,339 | (23.5) | 256,346 | (33.7) | 275,673 | (33.8) |

HYPERTENSION
(401)

Temporal Evaluation of FIB-4 vs. The Model of the Present Embodiments

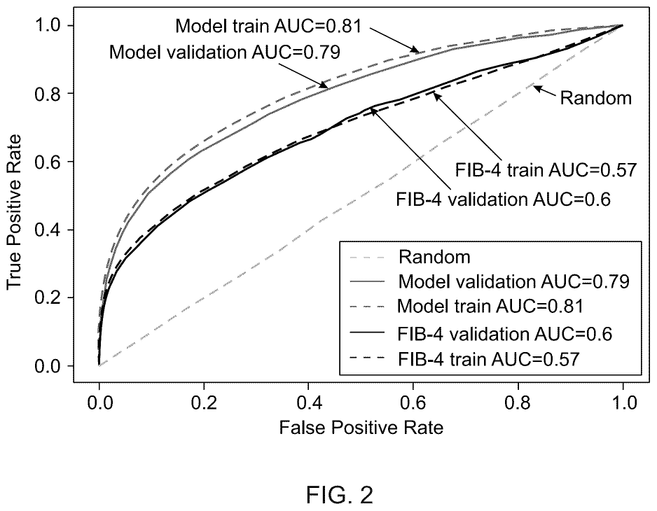

Data from the first two periods (2005-2010 and 2010-2015) was used for training a machine learning model predicting five-year follow-up outcome. A c-index of 0.80 (95% CI 0.80±6E-5) was observed on the train periods, and of 0.79 (95% CI 0.79±9E-5) on the validation period (2015-2020). For samples with available data (89.3%) FIB-4 score was calculated, and its c-index was 0.71 (95% CI 0.71±3E-4) on the train periods and 0.71 (95% CI 0.71±4E-4) on the validation period. Receiver operating characteristic curves (ROC) of both the model and FIB-4 score, over the training and validation periods (FIG. 2), show superior performance of the model over the FIB-4 risk score.

FIG. 2 shows evaluation on retrospective cohort. Shown are the false positive rate (x-axis) vs. the true positive rate (y-axis) curves of performance for the prediction model in orange and FIB-4 risk score in blue. Dashed lines show the performances on the train periods and continuous lines on the validation period. Area under the curve (AUC) for each curve is specified in the legend. As shown, the model obtained an area under the ROC (AUC) of 0.81 for the train periods and AUC of 0.79 for the validation period, while the FIB-4 score had an AUC of 0.58 for the train periods and AUC of 0.60 for the validation period. The Negative Predictive Values (NPV) and Positive Predictive Values (PPV) give set TPR and False Negative Rates (FNR) for the FIB-4 and for the model on the validation period were also computed and are provided in Tables 8 and 9, below. In Table 8, the FNR values were set to range from 10% to 90% at jumps of 10% each time, and the validation TPR, NPV and PPV were computed for both the model and the FIB-4 score. In Table 9, the TPR values were set to range from 10% to 90% at jumps of 10% each time, and the validation FNR, NPV and PPV were computed for both the model and the FIB-4 score. All values displayed in Tables 8 and 9 are percentages.

TABLE 8

| Specificity | Sensitivity (TPR) | | NPV | | PPV | |
|---|---|---|---|---|---|---|
| (1-FNR) | FIB-4 | Model | FIB-4 | Model | FIB-4 | Model |
| 10 | 93.7 | 98.1 | 99.7 | 99.9 | 0.5 | 0.6 |
| 20 | 89.3 | 96.2 | 99.7 | 99.9 | 0.6 | 0.6 |
| 30 | 84.9 | 93.3 | 99.7 | 99.9 | 0.6 | 0.7 |
| 40 | 79.6 | 89.4 | 99.7 | 99.9 | 0.7 | 0.8 |
| 50 | 74.1 | 84.8 | 99.7 | 99.8 | 0.7 | 0.9 |
| 60 | 66.4 | 79.1 | 99.7 | 99.8 | 0.8 | 1 |
| 70 | 59.5 | 71.5 | 99.7 | 99.8 | 1 | 1.2 |
| 80 | 50.7 | 63.7 | 99.7 | 99.8 | 1.3 | 1.6 |
| 90 | 39 | 51.2 | 99.7 | 99.7 | 1.9 | 2.5 |

TABLE 9

| Sensitivity | Specificity (1-FNR) | | NPV | | PPV | |
|---|---|---|---|---|---|---|
| (TPR) | FIB-4 | Model | FIB-4 | Model | FIB-4 | Model |
| 10 | 99.8 | 99.9 | 99.5 | 99.5 | 17 | 31.4 |
| 20 | 98.8 | 99.3 | 99.6 | 99.6 | 7.9 | 13.3 |
| 30 | 95.7 | 97.8 | 99.6 | 99.6 | 3.4 | 6.4 |
| 40 | 89.4 | 95.2 | 99.7 | 99.7 | 1.9 | 4 |
| 50 | 80.9 | 90.7 | 99.7 | 99.7 | 1.3 | 2.7 |
| 60 | 69.3 | 83.4 | 99.7 | 99.8 | 1 | 1.8 |
| 70 | 55.5 | 72.1 | 99.7 | 99.8 | 0.8 | 1.3 |
| 80 | 39.4 | 58.2 | 99.7 | 99.8 | 0.7 | 1 |
| 90 | 17.9 | 38.9 | 99.7 | 99.9 | 0.6 | 0.7 |

### Prospective External Cohort—Demographic Characteristics

Age, gender and latest laboratory test results (as of December 2021) were obtained from 115,211 Afula subdistrict's Clalit members (see Methods). Of those, 90,136 individuals passed inclusion exclusion criteria. This cohort had similar demographic characteristics to that of the retrospective cohort; where mean age was 56.9 (SD=9.9) and 55.2% of individuals were female (Table 6, above).

Prospective Evaluation of FIB-4 vs. The Model of the Present Embodiments

Both FIB-4 and model's scores were computed for the eligible (Table 3, above) 90,136 individuals (2.5% of which had missing data, and could not receive a FIB-4 score). To maintain a ratio of 1:3, a pooled sample of 435 individuals that obtained the highest risk score according to the model (referred to as model group) and 144 individuals with highest FIB-4 scores (FIB-4≥ 3.86, referred to as FIB-4 group) were included. There was no intersection between individuals in the model and FIB-4 group. To maintain a double-blinded study, FIB-4 and model scores were hidden from clinicians and study coordinators. Of the 579 individuals, 103 accepted enrollments to the prospective study, arriving at the clinic and undergoing a non-invasive physical examination (76 from model group, and 27 from FIB-4 group) (FIG. 3). 33/579 individuals passed away prior to recruitment (28/435 from model group and 5/144 from FIB-4 group, corresponding to 6.4% and 3.5%, respectively).

TE examinations of the 103 participants showed a significant difference in the mean liver stiffness of the model group kPa=13.1 versus the FIB-4 group kPa=6.5 (two-sided Mann-Whitney P<10⁻²)

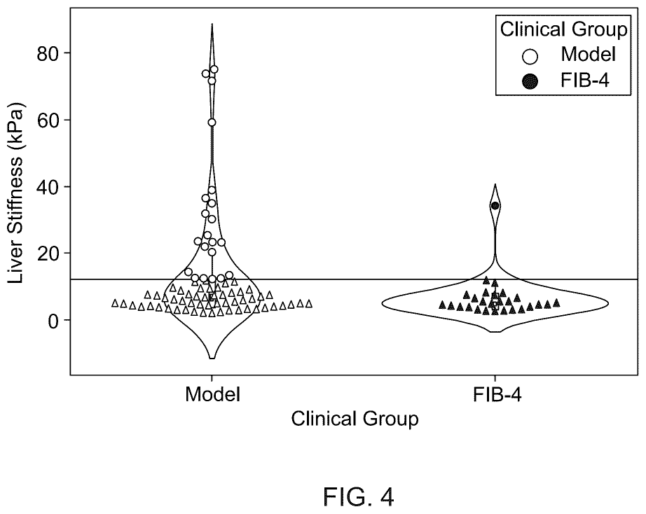

FIG. 4 shows liver stiffness in the clinical cohort. Those who arrived due to high model's predicted risk-scores are on the left and those who arrived due to high FIB-4 scores are on the right. The cutoff for liver cirrhosis is defined as kPa>12 (red line) and individuals marked as having liver cirrhosis appear as circles, whereas those who do not are marked as triangles.

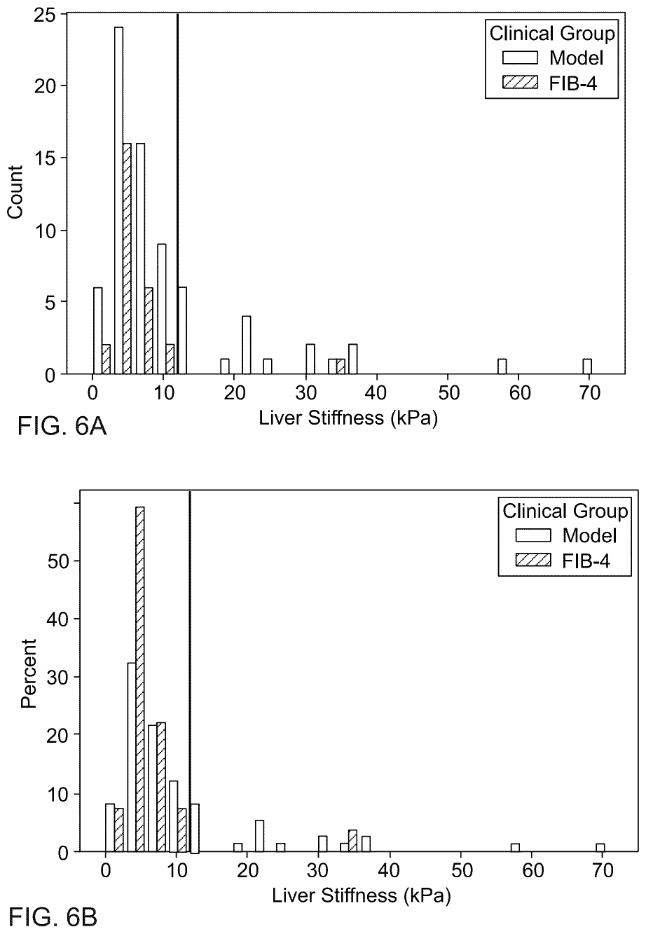

FIGS. 6A and 6B show counts (FIG. 6A) and percentage (FIG. 6B) of liver stiffness in the prospective cohort. Number of individuals by liver stiffness in the clinical trial separated by origin groups (high model's predicted risk in orange and high FIB-4 in blue). Liver cirrhosis disease kPa (>12) cutoff is marked in red.

Of the 103 participants, 21 (27.6%) from the model group were confirmed as having liver cirrhosis compared to only one (3.7%) from the FIB-4 group. The participant from the FIB-4 group, found to have cirrhosis, was the only participant with a world health organization (WHO) Alcohol Use Disorders Identification Test (AUDIT) 14 score above 15, indicating a moderate-severe alcohol use disorder. This participant was never diagnosed with alcohol abuse and therefore not excluded from the study.

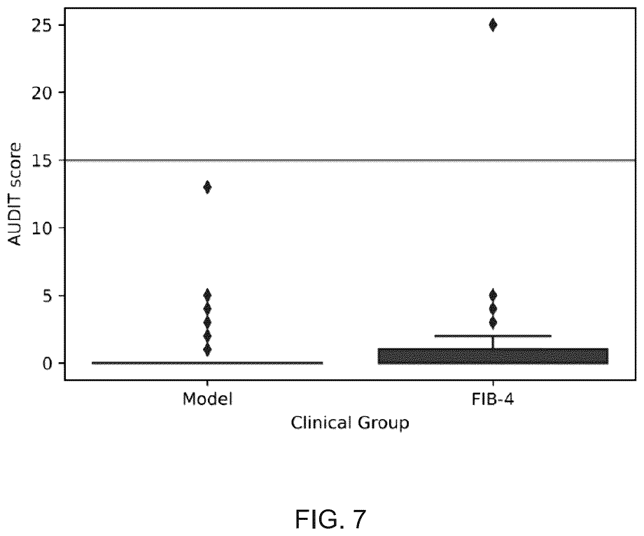

FIG. 7 shows AUDIT scores for individuals who arrived at the clinic separated by groups (high predicted risk on the left and high FIB-4 on the right). The threshold for moderate-severe alcohol use disorder is marked in red (15).

### Risk Factor Analysis

Risk factors were investigated by analyzing which features attribute to the model's prediction. To this end, the SHAP (SHapley Additive explanation) method (Lundberg et al., arXiv:1705.07874, 2017) was used. The SHAP interprets the output of a machine learning model. A SHAP value of a given parameter represents the average change in the model's output by conditioning on that particular parameter when introducing parameters one at a time over all parameter orderings. SHAP values were calculated individually for every parameter listed in Table 4, and are summarized in Table 10, below, ordered according to the calculated SHAP values.

TABLE 10

| | |
|---|---|
| AST | 0.977 |
| PLT | 0.340 |
| AGE | 0.280 |
| ALBUMIN | 0.254 |
| GLUCOSE | 0.102 |
| HB | 0.100 |
| HBA1C | 0.086 |
| PT | 0.078 |
| DAYS FROM BLOOD TEST | 0.073 |
| TOTAL PROTEIN | 0.072 |
| BILIRUBIN | 0.049 |
| CHOLESTEROL TOTAL | 0.031 |
| HDL | 0.030 |
| WBC | 0.030 |
| VITAMIN B12 | 0.027 |
| LDL | 0.023 |
| ALT | 0.016 |
| TRIGLYCERIDES | 0.007 |
| IS MALE | 0.006 |

### DISCUSSION

This Example described a retrospective study from nationwide EHR, to evaluate the effectiveness of implementing the machine learning model of the present embodiments into liver cirrhosis screening. This Example also prospective study that validates the potential of implementing the machine learning model of the present embodiments in comparison to the conventional FIB-4 score.

Liver diseases have been highlighted in recent calls for action [13,15,16] as well as in a recent EU health policy keynote statement [17]. Aiming at changing the diagnostic approach from late-stage patients to fibrosis diagnosis, previous studies have evaluated the potential of population-wide screening through FIB-4 or nonalcoholic fatty liver disease fibrosis scores [12,18]. These studies demonstrate that existing scores are likely insufficient for cost-effective screening of the general population.

In most real-world data, and especially in EHR data, missing information is often a major confounder of disease identification. Some existing risk scores cannot be calculated without available lab test results, and require repeated measurements for the continuous effectiveness of their population-wide potential [18]. Recent works, such as the SEAL project [19] show improved detection of liver cirrhosis; these too, however, demand active measurements of all individuals in the cohort. The technique of the present embodiments, which is based on real world EHR, can be continuously implemented on the entire population without the need for proactive routine checkups, as it allows for missing data and implementation at near zero cost. Due to its non-discrete nature, the technique of the present embodiments can be used for prioritization allowing policy decision makers to evaluate the cost-effectiveness of its implementation for different parts of the population.

The external prospective validation of the model's precision shoed that 27.6% individuals were diagnosed for the first time as having liver cirrhosis, compared to only 3.7%, a single patient of the control group based on FIB-4 risk score.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section 10 headings are used, they should not be construed as necessarily limiting. In addition, any priority document(s) of this application is/are hereby incorporated herein by reference in its/their entirety.

### REFERENCES

[1] Harrell F E, Califf R M, Pryor D B, Lee K L, Rosati R A. Evaluating the yield of medical tests. JAMA 1982; 247 (18): 2543-6.
[2] Asrani S K, Devarbhavi H, Eaton J, Kamath P S. Burden of liver diseases in the world. J Hepatol 2019; 70 (1): 151-71.
[3] Taylor R S, Taylor R J, Bayliss S, et al. Association Between Fibrosis Stage and Outcomes of Patients With Nonalcoholic Fatty Liver Disease: A Systematic Review and Meta-Analysis. Gastroenterology 2020; 158 (6): 1611-1625.e12.
[4] Sanyal A J, Van Natta M L, Clark J, et al. Prospective Study of Outcomes in Adults with Nonalcoholic Fatty Liver Disease. N Engl J Med 2021; 385 (17): 1559-69.
[5] Jepsen P, Ott P, Andersen P K, Sørensen H T, Vilstrup H. Clinical course of alcoholic liver cirrhosis: a Danish population-based cohort study. Hepatology 2010; 51 (5): 1675-82.
[6] Fleming K M, Aithal G P, Card T R, West J. The rate of decompensation and clinical progression of disease in people with cirrhosis: a cohort study. Aliment Pharmacol Ther 2010; 32 (11-12): 1343-50.
[7] Eddowes P J, Sasso M, Allison M, et al. Accuracy of fibroscan controlled attenuation parameter and liver stiffness measurement in assessing steatosis and fibrosis in patients with nonalcoholic fatty liver disease. Gastroenterology 2019; 156 (6): 1717-30.
[8] Hagström H, Talbäck M, Andreasson A, Walldius G, Hammar N. Ability of noninvasive scoring systems to identify individuals in the population at risk for severe liver disease. Gastroenterology 2020; 158 (1): 200-14.
[9] Harman D J, Ryder S D, James M W, et al. Obesity and type 2 diabetes are important risk factors underlying previously undiagnosed cirrhosis in general practice: a cross-sectional study using transient elastography. Aliment Pharmacol Ther 2018; 47 (4): 504-15.
[10] Angulo P, Hui J M, Marchesini G, et al. The NAFLD fibrosis score: a noninvasive system that identifies liver fibrosis in patients with NAFLD. Hepatology 2007; 45 (4): 846-54.
[11] Sterling R K, Lissen E, Clumeck N, et al. Development of a simple noninvasive index to predict significant fibrosis in patients with HIV/HCV coinfection. Hepatology 2006; 43 (6): 1317-25.
[12] Graupera I, Thiele M, Serra-Burriel M, et al. Low Accuracy of FIB-4 and NAFLD Fibrosis Scores for Screening for Liver Fibrosis in the Population. Clin Gastroenterol Hepatol 2022; 20 (11): 2567-2576.e6.
[13] Ginès P, Castera L, Lammert F, et al. Population screening for liver fibrosis: Toward early diagnosis and intervention for chronic liver diseases. Hepatology 2022; 75 (1): 219-28.
[14] Saunders J B, Aasland O G, Babor T F, de la Fuente J R, Grant M. Development of the Alcohol Use Disorders Identification Test (AUDIT): WHO Collaborative Project on Early Detection of Persons with Harmful Alcohol Consumption—II. Addiction 1993; 88 (6): 791-804.
[15] Ginès P, Graupera I, Lammert F, et al. Screening for liver fibrosis in the general population: a call for action. Lancet Gastroenterol Hepatol 2016; 1 (3): 256-60.
[16] Graupera I, Lammert F. Screening is caring: Community-based non-invasive diagnosis and treatment strategies for hepatitis C to reduce liver disease burden. J Hepatol 2018; 69 (3): 562-3.
[17] Karlsen T H, Sheron N, Zelber-Sagi S, et al. The EASL-Lancet Liver Commission: protecting the next generation of Europeans against liver disease complications and premature mortality. Lancet 2022; 399 (10319): 61-116.
[18] Hagström H, Talbäck M, Andreasson A, Walldius G, Hammar N. Repeated FIB-4 measurements can help identify individuals at risk of severe liver disease. J Hepatol 2020; 73 (5): 1023-9.
[19] Labenz C, Arslanow A, Nguyen-Tat M, et al. Structured Early detection of Asymptomatic Liver Cirrhosis: Results of the population-based liver screening program SEAL. J Hepatol 2022; 77 (3): 695-701.
[20] Rayan-Gharra N, Balicer R D, Tadmor B, Shadmi E. Association between cultural factors and readmissions: the mediating effect of hospital discharge practices and care-transition preparedness. BMJ Qual Saf 2019; 28 (11): 866-74.
[21] Slee V N. The International Classification of Diseases: ninth revision (ICD-9). Ann Intern Med 1978; 88 (3): 424-6.
[22] Bergmeir C, Benítez J M. On the use of cross-validation for time series predictor evaluation. Inf Sci (Ny) 2012; 191:192-213.
[23] Chen T, Guestrin C. XGBoost: A Scalable Tree Boosting System. In: Proceedings of the 22nd ACM SIGKDD International Conference on Knowledge Discovery and Data Mining—KDD '16. New York, New York, USA: ACM Press; 2016. p. 785-94.

1. A system for predicting probability for developing a liver associated disease, the system comprising a data processor having a circuit configured to obtain a plurality of parameters extracted from a body liquid test applied to a healthy subject, to access a computer readable medium storing a machine learning procedure trained for predicting probabilities for liver associated disease, to feed said procedure with said plurality of parameters, and to receive from said procedure an output indicative of a probability that said subject is expected to develop a liver associated disease.

2. The system according to claim 1, wherein said circuit is configured to receive from said procedure an output indicative of an expected onset time of said liver associated disease in said subject.

3. The system according to claim 1, wherein said plurality of parameters comprises a blood level of aspartate aminotransferase and a platelet score, and is devoid of blood hyaluronic acid concentration, and blood procollagen III-NP concentration.

4. (canceled)

5. A method of predicting probability for developing a liver associated disease, comprising:
obtaining a plurality of parameters extracted from a body liquid test applied to a healthy subject;
accessing a computer readable medium storing a machine learning procedure trained for predicting probabilities for liver associated disease;
feeding said procedure with said plurality of parameters; and
receiving from said procedure an output indicative of a probability that said subject is expected to develop a liver associated disease.

6. The method according to claim 5, comprising when said probability is above a predetermined threshold, performing at least one imaging test directed to identify the liver associated disease.

7. (canceled)

8. The method according to claim 5, comprising when said probability is above a predetermined threshold, periodically monitoring said subject for onset of the liver associated disease, by at least one technique selected from the group consisting of biopsy, MRI elastography, transient elastography, ultrasound elastography, and a fibrosis test.

9. The method according to claim 8, wherein said fibrosis test comprises at least one of: fibrosis-4 (FIB-4), aspartate aminotransferase to platelet ratio index (APRI), and aspartate aminotransferase to alanine aminotransferase ratio.

10. The method according to claim 5, wherein said output is indicative of an expected onset time of said liver associated disease in said subject.

11. The method according to claim 10, further comprising, a predetermined time ahead of said expected onset time, periodically monitoring said subject for onset of the liver associated disease, by at least one technique selected from the group consisting of biopsy, MRI elastography, transient elastography, ultrasound elastography, and a fibrosis test.

12. The method according to claim 5, wherein said plurality of parameters comprises a blood level of aspartate aminotransferase and a platelet score, and is devoid of blood hyaluronic acid concentration, and blood procollagen III-NP concentration.

13. The method according to claim 5, further comprising applying measures to prevent a development of the liver associated disease or to reduce said probability.

14-15. (canceled)

16. The method according to claim 13, wherein said measures comprise at least one of: administering a pharmaceutical agent, administering nutritional supplements or a dietary formulation, and providing recommendations for a change of life style and/or dietary habits.

17. A computer software product, comprising a computer-readable medium in which program instructions are stored, which instructions, when read by a data processor, cause the data processor to receive a food to which a response of a subject is unknown, and to execute the method according to claim 5.

18. A method of evaluating efficacy of a treatment for a liver associated disease, comprising:
obtaining a plurality of parameters extracted from a body liquid test applied to a subject undiagnosed with any liver associated condition;
accessing a computer readable medium storing a machine learning procedure trained for predicting probabilities for liver associated disease;
feeding said procedure with said plurality of parameters;
receiving from said procedure an output indicative of a probability that said subject is expected to develop a liver associated disease;
treating said subject by said treatment to evaluate the efficacy thereof only if said probability is above a predetermined threshold.

19. (canceled)

20. The system according claim 1, wherein said plurality of parameters comprises at least one parameter extracted from an electronic health record associated with said subject.

21. The system according to claim 1, wherein said plurality of parameters comprises at least one parameter extracted from a body liquid test applied to said subject.

22. The system er method according to claim 5, wherein said plurality of parameters comprises at least five blood levels or counts selected from the group consisting of: hemoglobin, platelet, white blood count, AST (GOT), ALT (GPT), albumin, bilirubin total, PT-INR, vitamin B12, glucose, hemoglobin A1c, cholesterol, cholesterol—HDL, cholesterol—LDL calc, triglycerides, and protein-total.

23. The system according to claim 5, wherein said plurality of parameters comprises blood level of aspartate aminotransferase, platelet score, an age of said subject, blood level of albumin, and blood level of glucose.

24. The system according to claim 5, wherein said machine learning procedure comprises at least one procedure selected from the group consisting of decision trees, clustering, support vector machine, linear modeling, k-nearest neighbors analysis, a set of decision trees, ensemble learning procedure, neural networks, probabilistic model, graphical model, Bayesian network, and association rule learning.

25-27. (canceled)

28. The method according to claim 5, wherein said liver associated disease comprises at least one of: liver cirrhosis, liver fibrosis, nonalcoholic fatty liver disease, non-alcoholic steatohepatitis, alcoholic fatty liver disease, alcoholic steatohepatitis, autoimmune hepatitis, hepatocarcinoma.
